(19) United States
(12) Patent Application Publication
(10) Pub. No.: US 2016/0001049 A1
(43) Pub. Date: Jan. 7, 2016

(54) BALLOON CATHETER

(71) Applicant: ASAHI INTECC CO., LTD., Nagoya-shi (JP)

(72) Inventors: Yuta NAKAGAWA, Osaka-shi (JP); Yuta KUBO, Seto-shi (JP)

(21) Appl. No.: 14/726,860

(22) Filed: Jun. 1, 2015

(30) Foreign Application Priority Data
Jul. 1, 2014 (JP) 2014-135567

(51) Int. Cl. A61M 25/10

(52) U.S. Cl. CPC A61M 25/1002; A61M 2025/1004

(57) ABSTRACT

A balloon catheter includes an inner tube and a balloon fixed to the inner tube. The balloon includes at least one first valley portion having a first distance to the inner tube, and at least one second valley portion having a second distance to the inner tube that is longer than the first distance. The first valley portion having the shorter distance to the inner tube can be preferentially depressurized for quick folding when the balloon transitions from an expanded state to a folded state. The second valley portion having the longer distance to the inner tube can be preferentially pressurized for quick expansion when the balloon transitions from a folded state to an expanded state.

### CROSS REFERENCE TO RELATED APPLICATION

This application claims priority to Japanese Application No. 2014-135567 filed on Jul. 1, 2014, the content of which is incorporated by reference herein in its entirety.

### BACKGROUND

The disclosed embodiments relate to a medical device. Specifically, the disclosed embodiments relate to a balloon catheter for securing the flow of fluid (e.g., blood, bile (gall), pancreatic fluid, or the like) through a lumen (e.g., a blood vessel, bile duct, pancreatic duct, or the like) by inserting the catheter into a stenosis site or obstructed segment formed in the lumen and expanding the stenosis site or obstructed segment.

When a stenosis site or an obstructed segment is formed in a lumen, the flow of fluid through the lumen becomes hindered. A balloon catheter has traditionally been used to treat such a stenosis site or an obstructed segment. A balloon catheter mainly comprises a balloon as an expandable body, an outer tube fixed to a proximal end of the balloon, and an inner tube inserted into the balloon and the outer tube. A guide wire can be inserted into the inner tube. An inflation lumen provided between the outer tube and the inner tube is used to pass a liquid (a contrast medium, physiological saline, or the like) to expand the balloon.

Usually, the balloon catheter is inserted into the lumen with the balloon folded around the outer periphery of the inner tube, and the balloon is expanded at the stenosis site or the obstructed segment. After treatment, the balloon is again folded around the outer periphery of the inner tube. The balloon therefore transitions from a folded state to an expanded state, and from an expanded state to a folded state. If there are multiple stenosis sites or obstructed segments, and the length of a stenosis site or an obstructed segment is longer than that of the balloon, the operator must repeat the above operations. As a result, if the balloon is not correctly shaped, the diameter of the balloon may not become small enough when folded, causing the operability of the balloon catheter to gradually decrease with repeat use.

To address this problem, a known balloon catheter includes a pre-shaped balloon having n (n is an integer of 2 or more) wing portions and n (n is an integer of 2 or more) valley portions formed between adjacent wing portions when folded around the outer periphery of the inner tube (for example, see Japanese Patent No. 4761671). However, distances between the n valley portions and the inner tube are all the same in the known balloon catheter. Therefore, pressurization or depressurization of the balloon exerts pressure evenly on each of the n valley portions when the balloon transitions from a folded state to an expanded state, or when the balloon transitions from an expanded state to a folded state. In particular, when the balloon catheter is inserted up to the end of a curved lumen, considerable time is required to fold the valley portions of the balloon during depressurization, or to expand the valley portions of the balloon during pressurization. Accordingly, it takes longer for the balloon to transition from a folded state to an expanded state, or from an expanded state to a folded state. The known balloon catheter therefore disadvantageously requires a long time to expand a stenosis site or an obstructed segment.

### SUMMARY

The disclosed embodiments have been devised in view of the circumstances described above. An object of the disclosed embodiments is to provide a balloon catheter in which a distance between at least one valley portion of n valley portions and an inner tube is shorter than distances between the other valley portions and the inner tube. The at least one valley portion having the shorter distance to the inner tube preferentially depressurizes when the balloon transitions from an expanded state to a folded state, shortening the time required to fold the balloon. The valley portions having the longer distance to the inner tube preferentially pressurize when the balloon transitions from a folded state to an expanded state, shortening the time required to expand the balloon.

The disclosed embodiments include a balloon catheter comprising an inner tube and a balloon fixed to the inner tube. The balloon is capable of transitioning between a folded state and an expanded state, and includes n (n is an integer of 2 or more) wing portions and n (n is an integer of 2 or more) valley portions formed between adjacent wing portions when the balloon is folded around an outer periphery of the inner tube. A distance between at least one valley portion of the n valley portions and the inner tube is shorter than distances between the other valley portions and the inner tube.

When the balloon having this configuration transitions from an expanded state to a folded state, the at least one valley portion having the shorter distance to the inner tube is depressurized more preferentially than the other valley portions having the longer distance to the inner tube. The at least one valley portion having the shorter distance to the inner tube therefore starts to fold first. This also triggers folding of the other valley portions having the longer distances to the inner tube, because the at least one valley portion having the shorter distance will drag the other valley portions toward the inner tube. Therefore, the other valley portions having the longer distances to the inner tube will start to fold even before a pressure is exerted on them by depressurization. This can shorten the time required for the balloon to transition from an expanded state to a folded state.

Similarly, when the balloon transitions from a folded state to an expanded state, the other valley portions having the longer distances to the inner tube are pressurized more preferentially than the at least one valley portion having the shorter distance to the inner tube. The other valley portions having the longer distance to the inner tube therefore start to expand first. This also triggers expansion of the at least one valley portion having the shorter distance to the inner tube, because the at least one valley portion having the shorter distance will be dragged away from the inner tube by the other valley portions. Therefore, the at least one valley portion having the shorter distance to the inner tube will start to expand even before a pressure is exerted on it by pressurization. This can shorten the time required for the balloon to transition from a folded state to an expanded state.

The balloon catheter may include n valley portions where a distance between an n-th valley portion and the inner tube is shorter than a distance between an (n−1)th valley portion adjacent to the n-th valley portion and the inner tube, and a distance between an (n−2)th valley portion adjacent to the (n−1)th valley portion and the inner tube is shorter than the distance between the (n−1)th valley portion and the inner tube. When the balloon transitions from an expanded state to a folded state, the n-th valley portion and the (n−2)th valley portion having the shorter distances to the inner tube are depressurized more preferentially than the (n−1)th valley portion having the longer distance to the inner tube, and therefore start to fold first. Because the (n−1)th valley portion is sandwiched between the n-th valley portion and the (n−2)th valley portion, it is dragged from both sides by the n-th valley portion and the (n−2)th valley portion during folding. As a result, the time required for the balloon to transition from an expanded state to a folded state can be further shortened.

The n valley portions of the balloon catheter may comprise a plurality of “short valley portions” and a plurality of “long valley portions.” The long valley portions have a longer distance to the inner tube than the short valley portions. The short valley portions and the long valley portions are alternately arranged in a circumferential direction. Therefore, when the balloon transitions from an expanded state to a folded state, the valley portions having the shorter distance to the inner tube will start to fold first, and will fold evenly in the circumferential direction (that is, will fold symmetrically around the outer periphery of the inner tube). The valley portions having the longer distance to the inner tube will each be dragged from both sides because each one is sandwiched between valley portions having the shorter distance to the inner tube. As a result, the balloon will fold symmetrically relative to the inner tube, and the time required for the balloon to transition from an expanded state to a folded state can be further shortened.

Similarly, when the balloon transitions from a folded state to an expanded state, the valley portions having the longer distance to the inner tube will start to expand first, and will expand evenly in the circumferential direction (that is, will expand symmetrically away from the outer periphery of the inner tube). The valley portions having the shorter distance to the inner tube will each be dragged from both sides because each one is sandwiched between valley portions having the longer distance to the inner tube. As a result, the balloon will expand symmetrically relative to the inner tube, and the time required for the balloon to transition from a folded state to an expanded state can be further shortened.

### BRIEF DESCRIPTION OF THE DRAWINGS

FIG. 1 shows an overall view of a balloon catheter (with a balloon expanded) according to the disclosed embodiments.

FIG. 2 is a cross-sectional view taken along line A-A of FIG. 1.

FIG. 3 shows an overall view of a balloon catheter (with the balloon folded) according to the disclosed embodiments.

FIG. 4 is a cross-sectional view taken along line B-B of FIG. 3.

FIGS. 5A-5D show how the balloon of FIG. 4 transitions from an expanded state to a folded state (or from a folded state to an expanded state). FIG. 5A shows a state where the balloon is expanded. FIG. 5B shows a state where a valley portion having a shorter distance to the inner tube starts to preferentially fold. FIG. 5C shows a state where valley portions having a longer distance to the inner tube start to fold by being dragged by the valley portion having the shorter distance. FIG. 5D shows a state where the 6 valley portions are all folded.

FIG. 6 is a cross-sectional view of a balloon of the disclosed embodiments in the folded state.

FIGS. 7A to 7D show how the balloon of FIG. 6 transitions from an expanded state to a folded state, or from a folded state to an expanded state.

FIG. 8 is a cross-sectional view of a balloon of the disclosed embodiments in the folded state.

FIGS. 9A to 9D show how the balloon of FIG. 8 transitions from an expanded state to a folded state, or from a folded state to an expanded state.

### DESCRIPTION OF EMBODIMENTS

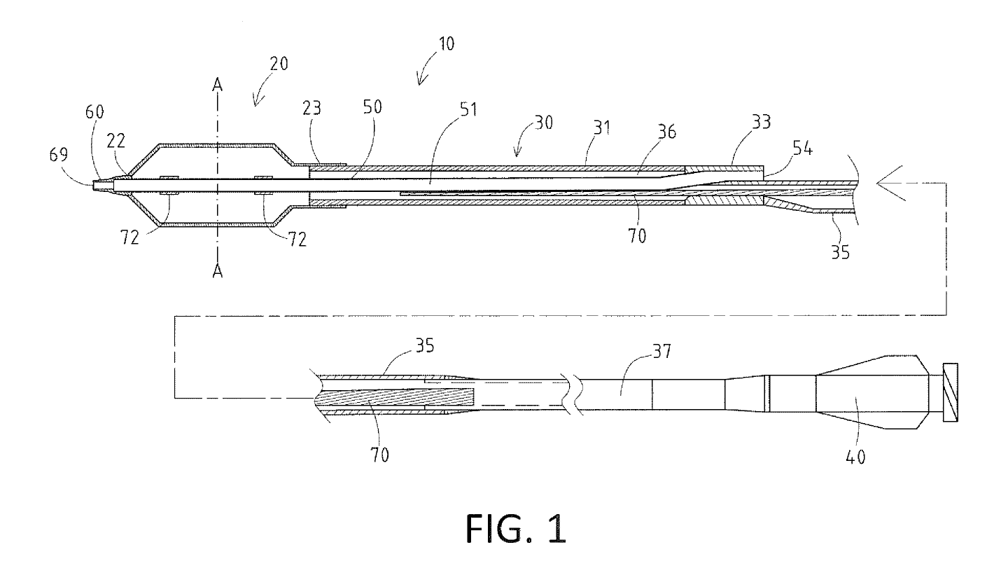

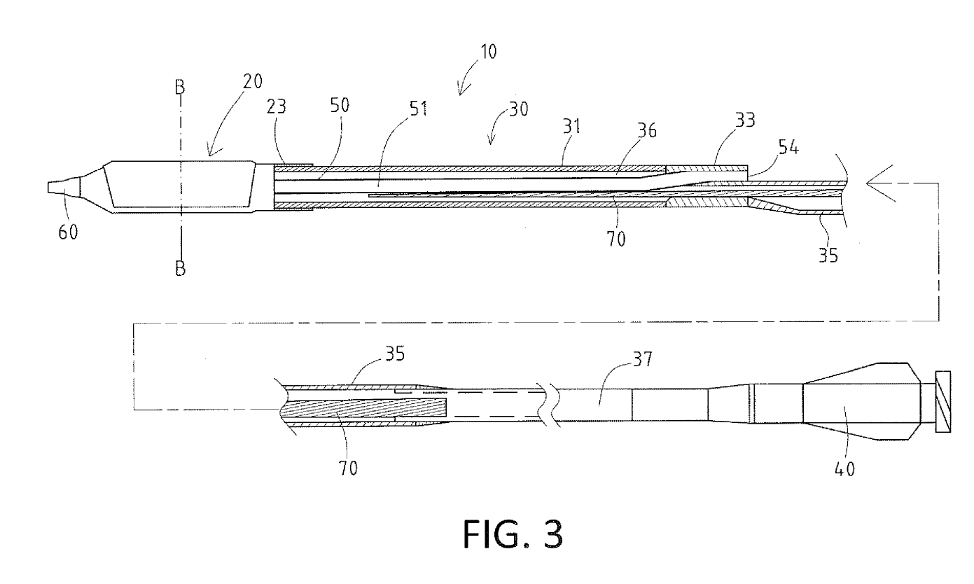

A balloon catheter 10 of the disclosed embodiments will be described with reference to FIGS. 1 to 5D. The left side in FIGS. 1 and 3 corresponds to the distal end (the front end), which is to be inserted into the body, and the right side corresponds to the proximal end (the base end), which is to be operated by an operator such as a physician.

The balloon catheter 10 may be used for treating, for example, a stenosis site or an obstructed segment formed in a lumen (e.g., a blood vessel, bile duct, pancreatic duct, or the like). As shown in FIG. 1, the balloon catheter 10 mainly comprises a balloon 20, an outer tube 30, a connector 40, an inner tube 50, a tip 60, and a reinforcer 70. Note that FIG. 1 shows the balloon 20 in an expanded state.

The balloon 20 for expanding a stenosis site or obstructed segment comprises a resin material, and has a distal attaching portion 22 at its distal end and a proximal attaching portion 23 at its proximal end. The distal attaching portion 22 is fixed to the tip 60 and the distal end of the inner tube 50, and the proximal attaching portion 23 is fixed to the distal end of the outer tube 30. Although the distal attaching portion 22 is fixed to the distal end of the inner tube 50 through the tip 60 in FIG. 1, the configuration is not limited to this. The distal attaching portion 22 may be sandwiched between the tip 60 and the distal end of the inner tube 50. Further, although the proximal attaching portion 23 is fixed to a distal end of the outer periphery of the outer tube 30 in FIG. 1, the configuration is not limited to this. The proximal attaching portion 23 may be fixed to a distal end of the inner periphery of the outer tube 30.

The outer tube 30 is a tubular member comprising an inflation lumen 36 for supplying a liquid such as a contrast medium or a physiological saline in order to expand the balloon 20. The outer tube 30 comprises a distal end outer tube part 31, a guide wire port part 33, a middle outer tube part 35, and a proximal end outer tube part 37 in that order from the distal end of the outer tube 30 to the proximal end of the outer tube 30. The distal end outer tube part 31 and the middle outer tube part 35 are tubes that may comprise a resin such as a polyamide, polyamide elastomer, polyolefin, polyester, or polyester elastomer. The distal end outer tube part 31, the middle outer tube part 35, and the inner tube 50 are fixed to the guide wire port part 33.

The inner tube 50 is inserted into the distal end outer tube part 31, and the aforementioned inflation lumen 36 is formed between the distal end outer tube part 31 and the inner tube 50.

The proximal end outer tube part 37 may be a metal tubular member called a “hypo tube.” The distal end of the proximal end outer tube part 37 is inserted into and fixed to the proximal end of the middle outer tube part 35. The connector 40 is attached to the proximal end of the proximal end outer tube part 37. When a liquid for expanding the balloon 20 is supplied from an indeflator (not shown) attachable to the connector 40, the liquid flows through the inflation lumen 36 to expand the balloon 20. Note that there is no particular limitation for the material of the proximal end outer tube part 37, and stainless steel (SUS304) or a superelastic alloy such as an Ni—Ti alloy can be used.

The inner tube 50 forms a guide wire lumen 51 for inserting a guide wire into the balloon catheter. Further, the proximal end of the inner tube 50 is fixed to the guide wire port part 33 of the outer tube 30 and forms a proximal side guide wire port 54.

The distal end of the inner tube 50 is fixed to the tip 60 and the distal attaching portion 22 of the balloon 20. The tip 60 has a tapered external shape in which the outer diameter gradually decreases toward the distal end of the tip 60, and may be formed with a flexible resin. There is no particular limitation for the resin for forming the tip 60, and a polyurethane, polyurethane elastomer, or the like can be used.

The tip 60 is a cylindrical member fixed to the distal end of the guide wire lumen 51, and has a distal guide wire port 69 at the distal end.

Two radiopaque markers 72 may be attached to the inner tube 50 inside the balloon 20 so that the position of the balloon 20 can be detected under radiation.

The reinforcer 70 is attached to a distal end of the inner periphery of the proximal end outer tube part 37. The reinforcer 70 has a circular cross section, and is a tapered metal wire member in which the diameter decreases toward the distal end of the reinforcer 70. There is no particular limitation for the material of the reinforcer 70, and stainless steel (SUS304) or a superelastic alloy such as an Ni—Ti alloy can be used.

The reinforcer 70 passes through the middle outer tube part 35 and the guide wire port part 33, and extends to the distal end outer tube part 31. The distal end of the reinforcer 70 may be fixed to the inner tube 50 as shown in FIG. 1, but the configuration is not limited to this. For example, the distal end of the reinforcer 70 may be fixed so that it is sandwiched between the outer tube 30 and the inner tube 50.

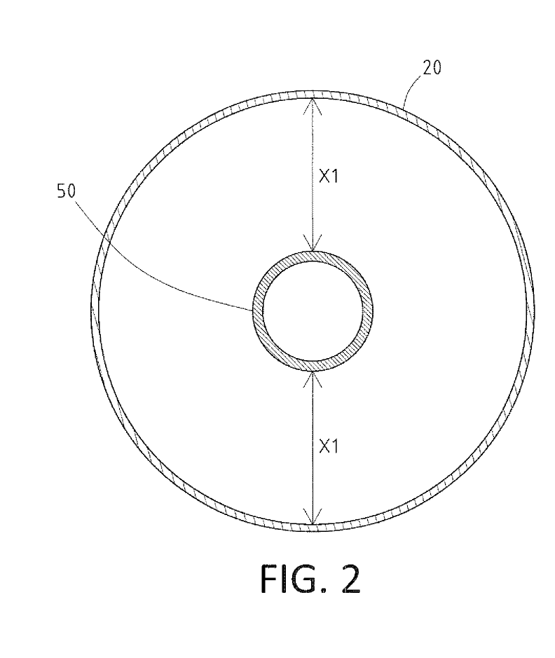

FIG. 2 is a cross-sectional view taken along line A-A of FIG. 1. As shown in FIG. 2, when a liquid such as a contrast medium or a physiological saline is supplied to the balloon 20, the balloon 20 is expanded outwardly around the circumference of the inner tube 50 so that the inner tube 50 is positioned at the center of the balloon 20. At this time, the distance between the balloon 20 and the inner tube 50 is defined as X1.

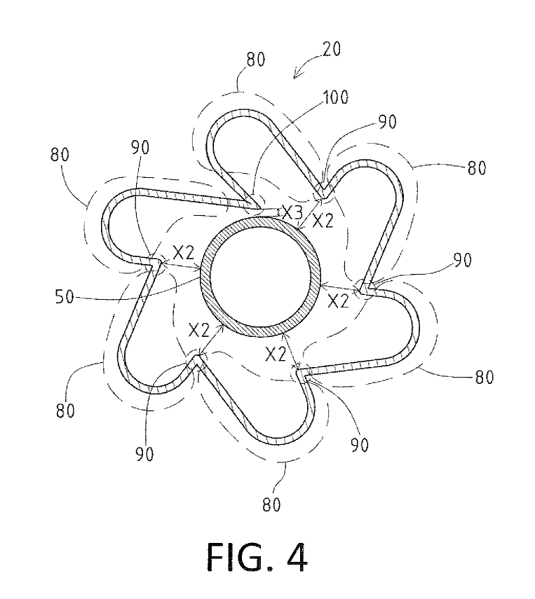

FIG. 3 shows a state in which the balloon 20 is folded around the outer periphery of the inner tube 50. Note that unlike in FIG. 1, a portion including the tip 60 and the balloon 20 is shown as an external view in FIG. 3 instead of a cross-sectional view for the purposes of illustration. FIG. 4 is a cross-sectional view along line B-B of FIG. 3. As shown in FIG. 4, the balloon 20 has 6 wing portions 80, and 6 valley portions 90, 100 formed between adjacent wing portions 80 when the balloon 20 is folded around the outer periphery of the inner tube 50. The 6 valley portions 90, 100 are grouped into two groups: 5 valley portions 90 having a “long distance” X2 to the inner tube 50, and one valley portion 100 having a “short distance” X3 to the inner tube 50. That is, the “long distance” X2 is longer than the “short distance” X3 (X2>X3).

To form the balloon 20, 6 valley portions 90 having a long distance X2 to the inner tube 50 are loosely shaped by loosely pressurizing the balloon 20 in a mold. Subsequently, a valley portion 100 having a short distance X3 to the inner tube 50 is produced by wrapping one valley portion 90 of the 6 valley portions 90 and performing physically firm shaping. That is, the valley portion 100 is formed by re-shaping one of the valley portions 90 to have a shorter distance to the inner tube 50. However, there is no particular limitation for the method of differentially producing the valley portions 90 and the valley portion 100. For example, a loosely shaped valley portion 90 having a long distance X2 to the inner tube 50 and a firmly shaped valley portion 100 having a short distance X3 to the inner tube 50 may be produced by pressurizing the balloon 20 in a mold already having the valley portions 90 having the long distance X2 to the inner tube 50 and the valley portion 100 having the short distance X3 to the inner tube 50.

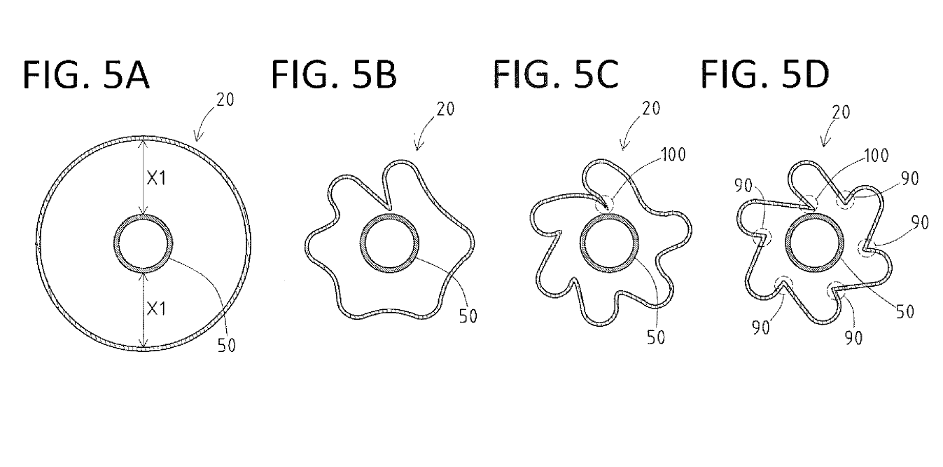

FIGS. 5A to 5D show how the balloon 20 transitions from an expanded state to a folded state. Note that the balloon 20 is described with reference to the valley portions 90, 100 as shown in FIGS. 5A to 5D for clear understanding. Therefore, description about the wing portions 80 is omitted. When the liquid supplied to the balloon 20 is removed with an indeflator, the balloon 20 is depressurized. When the liquid is removed, the firmly shaped valley portion 100 is depressurized more preferentially than the loosely shaped valley portions 90, and starts to fold first (see FIG. 5B). The loosely shaped valley portions 90 are then dragged by the valley portion 100 when the valley portion 100 starts to fold (see FIG. 5C).

As described above, the balloon 20 has the loosely pre-shaped valley portions 90 having the long distance X2 to the inner tube 50 and the firmly pre-shaped valley portion 100 having the short distance X3 to the inner tube 50 (see FIG. 4). As a result, the valley portion 100 preferentially folds and drags the valley portions 90 toward the inner tube 50 when the balloon 20 is depressurized. This reduces the time required for the balloon 20 to transition from an expanded state to a folded state because the valley portions 90 start to fold before a pressure is applied to them by depressurization.

Further, the balloon 20 transitions from a folded state to an expanded state (in other words, the balloon 20 transitions from a state shown in FIG. 5D to a state shown in FIG. 5A) as the balloon 20 is pressurized by the liquid supplied to the balloon 20 with the indeflator. When the liquid is supplied, the loosely shaped valley portions 90 are pressurized more preferentially than the firmly shaped valley portion 100, and start to expand first (see FIG. 5C). When the valley portions 90 start to expand, the firmly shaped valley portion 100 is dragged by the valley portions 90 (see FIG. 5B).

As described above, the balloon 20 has the loosely pre-shaped valley portions 90 having the long distance X2 to the inner tube 50 and the firmly pre-shaped valley portion 100 having the short distance X3 to the inner tube 50 (see FIG. 4). As a result, the valley portions 90 preferentially expand and drag the valley portion 100 away from the inner tube 50 when the balloon 20 is pressurized. This reduces the time required for the balloon 20 to transition from a folded state to an expanded state because the valley portion 100 starts to expand before a pressure is applied to it by pressurization.

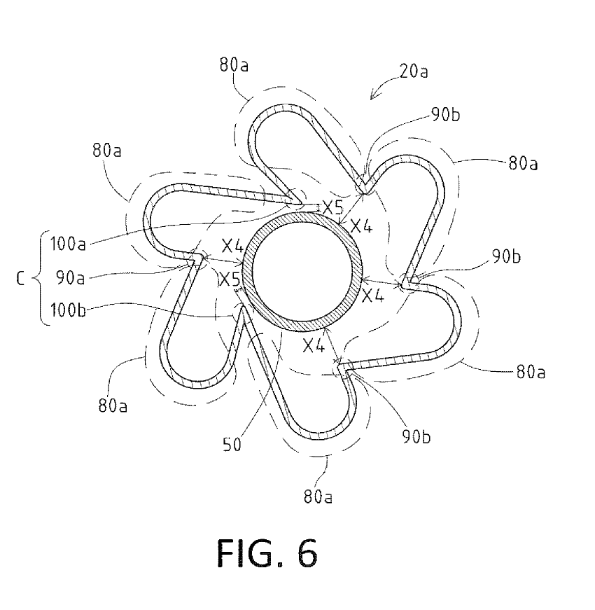

FIG. 6 shows a balloon 20a of the disclosed embodiments. When the balloon 20a is folded around the outer periphery of the inner tube 50, the balloon 20a has 6 wing portions 80a and 6 valley portions 90a, 90b, 100a, 100b formed between adjacent wing portions 80a. The 6 valley portions 90a, 90b, 100a, 100b are grouped into two groups: 4 valley portions 90a, 90b having a “long distance” X4 to the inner tube 50, and 2 valley portions 100a, 100b having a “short distance” X5 to the inner tube 50. That is, the “long distance” X4 is longer than the “short distance” X5 (X4>X5).

The balloon 20 shown in FIG. 4 has only one valley portion 100 having the short distance X3 to the inner tube 50. In contrast, the balloon 20a shown in FIG. 6 has 2 valley portions 100a, 100b having the short distance X5 to the inner tube 50, and the valley portion 90a having the long distance X4 to the inner tube 50 is provided between the 2 valley portions 100a, 100b. Therefore, when the balloon 20a is folded around the outer periphery of the inner tube 50, the balloon 20a has a portion where the distance X5 from the valley portion 100a to the inner tube 50 is shorter that the distance X4 from the adjacent valley portion 90b to the inner tube 50, and the distance X5 from the valley portion 100b adjacent the valley portion 90a to the inner tube 50 is shorter than the distance X4 from the valley portion 90a to the inner tube 50 (see Part C in FIG. 6).

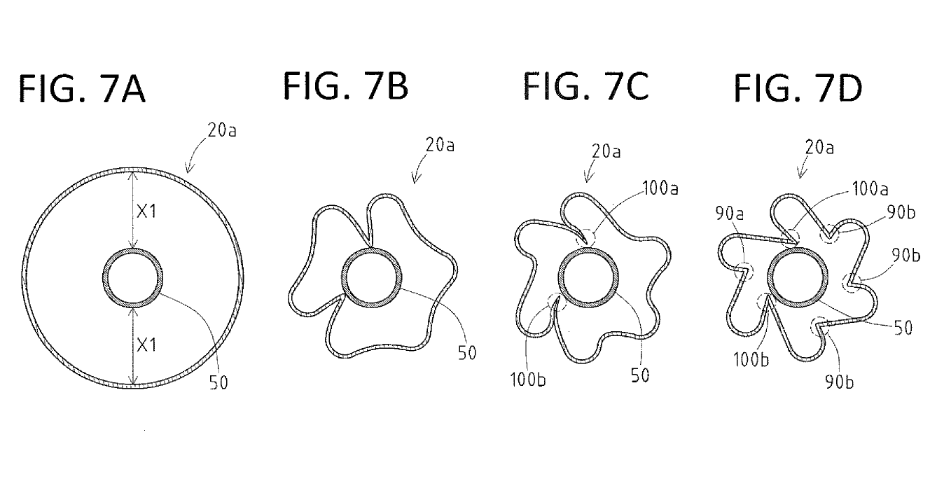

FIGS. 7A to 7D show how the balloon 20a transitions from an expanded state to a folded state. Note that the balloon 20a is described with reference to the valley portions 90a, 90b, 100a, 100b of the balloon 20a as shown in FIGS. 7A to 7D for clear understanding. Therefore, description about the wing portions 80a is omitted. When the liquid supplied to the balloon 20a is removed with an indeflator, the balloon 20a is depressurized. When the liquid is removed, the firmly shaped valley portions 100a and 100b are depressurized more preferentially than the loosely shaped valley portions 90a, 90b and start to fold first (see FIG. 7B). The valley portion 90a starts to fold by being dragged from both sides by the valley portions 100a and 100b because the valley portion 90a is sandwiched between the valley portions 100a and 100b (see FIG. 7C).

Therefore, the valley portions 100a and 100b preferentially fold and drag the valley portion 90a from both sides toward the inner tube 50 when the balloon 20a is depressurized. This reduces the time required for the balloon 20a to transition from an expanded state to a folded state because the valley portion 90a starts to fold before a pressure is applied to it by depressurization.

Further, the balloon 20a transitions from a folded state to an expanded state (in other words, the balloon 20a transitions from a state shown in FIG. 7D to a state shown in FIG. 7A) as the balloon 20a is pressurized by the liquid supplied to the balloon 20a with the indeflator. When the liquid is supplied, the loosely shaped valley portions 90a, 90b are pressurized more preferentially than the firmly shaped valley portions 100a and 100b, and start to expand first (see FIG. 7C). When the valley portions 90a, 90b start to expand, the valley portions 100a and 100b are dragged away from the inner tube 50 at both sides (see FIG. 7B).

In this way, the valley portions 90a, 90b preferentially expand and drag the valley portions 100a and 100b away from the inner tube 50 when the balloon 20a is pressurized. This reduces the time required for the balloon 20a to transition from a folded state to an expanded state because the valley portions 100a and 100b start to expand before a pressure is applied to them by pressurization.

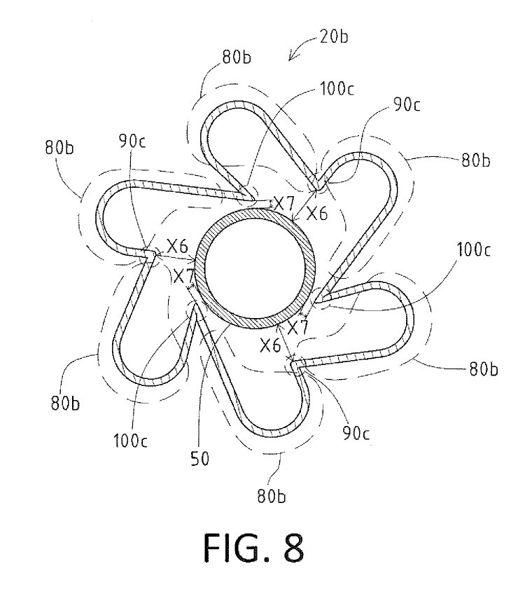

FIG. 8 shows a balloon 20b of the disclosed embodiments that has 6 wing portions 80b and 6 valley portions 90c, 100c formed between adjacent wing portions 80b when the balloon 20b is folded around the outer periphery of the inner tube 50. The 6 valley portions 90c, 100c are grouped into two groups: 3 long valley portions 90c having a “long distance” X6 to the inner tube 50 and 3 short valley portions 100c having a “short distance” X7 to the inner tube 50. That is, the “long distance” X6 is longer than the “short distance” X7 (X6>X7). The 3 long valley portions 90c and the 3 short valley portions 100e are alternately arranged in the circumferential direction (i.e., around the outer periphery of the inner tube 50).

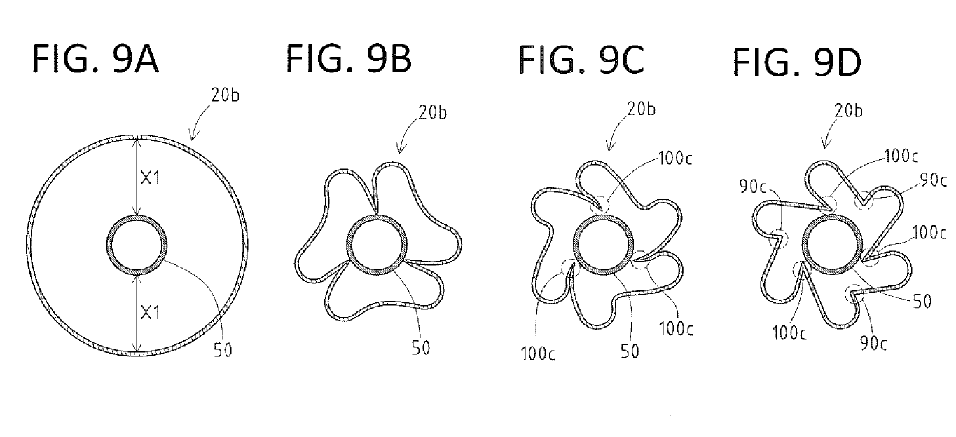

FIGS. 9A to 9D show how the balloon 20b transitions from an expanded state to a folded state. Note that the balloon 20b is described with reference to the valley portions 90c, 100c of the balloon 20b as shown in FIGS. 9A to 9D for clear understanding. Therefore, description about the wing portions 80b is omitted. When the liquid supplied to the balloon 20b is removed with the indeflator, the balloon 20b is depressurized. When the liquid is removed, the firmly shaped short valley portions 100c are depressurized more preferentially than the loosely shaped long valley portions 90c, and start to fold first (see FIG. 9B). The short valley portions fold evenly in a circumferential direction (i.e., symmetrically around the outer periphery of the inner tube 50). The loosely shaped long valley portions 90c are dragged from both sides toward the inner tube 50 by the short valley portions 100c when the short valley portions 100e start to fold (see FIG. 9C). Thus, the long valley portions also fold evenly in a circumferential direction.

Therefore, the short valley portions 100e preferentially and evenly fold and drag the long valley portions 90c sandwiched between the short valley portions 100c from both sides toward the inner tube 50 when the balloon 20b is depressurized. As a result, the balloon 20b will fold symmetrically relative to the inner tube 50, and the time required for the balloon 20b to transition from an expanded state to a folded state can be shortened because the long valley portions 90c start to fold evenly before a pressure is applied to them by depressurization.

Further, the balloon 20b transitions from a folded state to an expanded state (in other words, the balloon 20b transitions from a state shown in FIG. 9D to a state shown in FIG. 9A) as the balloon 20b is pressurized by the liquid supplied to the balloon 20b with the indeflator. When the liquid is supplied, the loosely shaped long valley portions 90c are pressurized more preferentially than the firmly shaped short valley portions 100c, and start to expand evenly in a circumferential direction (see FIG. 9C). When the short valley portions 90c start to expand, the firmly shaped short valley portions 100e are dragged away from the inner tube 50 from both sides by the long valley portions 90c (see FIG. 9B).

As described above, the long valley portions 90c preferentially and evenly expand and drag the short valley portions 100c sandwiched between the long valley portions 90c from both sides away from the inner tube 50 when the balloon 20b is pressurized. As a result, the balloon 20b will unfold symmetrically relative to the inner tube 50, and the time required for the balloon 20b to transition from a folded state to an expanded state can be further shortened because the short valley portions 100c start to expand evenly before a pressure is applied to them by pressurization.

Note that the balloons 20, 20a, 20b are described as having 6 wing portions 80, 80a, 80b and 6 valley portions 90, 90a, 90b, 90c, 100, 100a, 100b, 100c when folded around the outer periphery of the inner tube 50 (see FIGS. 4, 6, and 8). However, there is no particular limitation for the number of the wing portions 80, 80a, 80b and the valley portions 90, 90a, 90b, 90c, 100, 100a, 100b, 100c.

For example, the balloon 20 may have n (n is an integer of 2 or more) wing portions 80 and n (n is an integer of 2 or more) valley portions 90, 100 formed between adjacent wing portions 80 when the balloon 20 is folded around the outer periphery of the inner tube 50, as long as the distance X3 from at least one valley portion 100 of the n valley portions 90, 100 to the inner tube 50 is shorter than the distances X2 from other valley portions 90 to the inner tube 50. Further, the balloon 20a may also have n wing portions and n valley portions, where the distance X5 between the n-th valley portion 100a and the inner tube 50 is shorter than the distance X4 between the adjacent (n−1)th valley portion 90a and the inner tube 50 when the balloon 20a is folded around the outer periphery of the inner tube 50, and the distance X5 between the (n−2)th valley portion 100b adjacent to the (n−1)th valley portion 90a and the inner tube 50 is shorter than the distance X4 between the (n−1)th valley portion 90a and the inner tube 50. The balloon 20b may have n wing portions and n valley portions, as long as the short valley portions 100c having the short distance X7 to the inner tube 50 and the long valley portions 90c having the long distance X6 to the inner tube 50 are alternately provided in a circumferential direction when the balloon 20b is folded around the outer periphery of the inner tube 50.

As described above, the distance between at least one valley portion 100, 100a, 100b, 100c of the n valley portions 90, 90a, 90b, 90c, 100, 100a, 100b, 100c and the inner tube 50 is shorter than distances between other valley portions 90, 90a, 90b, 90c and the inner tube 50 when the balloons 20, 20a, 20b of the balloon catheter 10 are folded around the outer periphery of the inner tube 50. As a result, the valley portions 100, 100a, 100b, 100c having a short distance to the inner tube 50 can be preferentially depressurized to quickly fold when the balloons 20, 20a, 20b transition from an expanded state to a folded state, while the valley portions 90, 90a, 90b, 90c having a long distance to the inner tube 50 can be preferentially pressurized to quickly expand when the balloons 20, 20a, 20b transition from a folded state to an expanded state.

What is claimed:

1. A balloon catheter comprising:
an inner tube; and
a balloon fixed to the inner tube and capable of transitioning between a folded state and an expanded state,
wherein, when the balloon is folded around an outer periphery of the inner tube:
the balloon has a plurality of wing portions and a plurality of valley portions, each valley portion being formed between adjacent wing portions, and
the plurality of valley portions comprise at least one first valley portion having a first distance between the first valley portion and the inner tube, and at least one second valley portion having a second distance between the second valley portion and the inner tube, the first distance being shorter than the second distance.

2. The balloon catheter according to claim 1, wherein:
the plurality of valley portions further comprise at least one third valley portion having a third distance between the third valley portion and the inner tube,
each of the at least one second valley portion is adjacent to and interposed between one of the at least one first valley portion and one of the at least one third valley portion, and
the third distance is shorter than the second distance.

3. The balloon catheter according to claim 1, wherein the plurality of valley portions comprise a plurality of first valley portions and a plurality of second valley portions.

4. The balloon catheter according to claim 3, wherein the plurality of first valley portions and the plurality of second valley portions are alternately arranged in a circumferential direction with respect to an outer periphery of the first inner tube.

5. The balloon catheter according to claim 4, wherein each of the plurality of first valley portions is adjacent to and interposed between two of the plurality of second valley portions.

6. The balloon catheter according to claim 2, wherein the plurality of valley portions comprise a plurality of the first valley portions, a plurality of the second valley portions, and a plurality of the third valley portions.

7. The balloon catheter according to claim 2, wherein the first distance and the third distance are the same.

8. The balloon catheter according to claim 1, wherein the balloon has at least six wing portions and at least six valley portions when the balloon is folded around an outer periphery of the inner tube.
